(19) United States
(12) Patent Application Publication
(10) Pub. No.: US 2011/0098482 A1
(43) Pub. Date: Apr. 28, 2011

(54) METHODS OF PREPARING 1-(4-((1R,2S,3R)-1,2,3,4-TETRAHYDROXYBUTYL)-1H-IMIDAZOL-2-YL)ETHANONE

(76) Inventor: Wenxue Wu, Princeton Junction, NJ (US)

(21) Appl. No.: 12/911,172

(22) Filed: Oct. 25, 2010

Related U.S. Application Data
(60) Provisional application No. 61/254,960, filed on Oct. 26, 2009.

(51) Int. Cl. C07D 233/64

(52) U.S. Cl. 548/333.5

(57) ABSTRACT

Methods of preparing 1-(4-((1R,2S,3R)-1,2,3,4-tetrahydroxybutyl)-1H-imidazol-2-yl)ethanone and derivatives thereof are disclosed.

This application claims priority to U.S. provisional application No. 61/254,960, filed Oct. 26, 2009, the entirety of which is incorporated herein by reference.

### 1. FIELD OF THE INVENTION

This invention relates to methods of synthesizing 1-(4-((1R,2S,3R)-1,2,3,4-tetrahydroxybutyl)-1H-imidazol-2-yl)ethanone and derivatives thereof.

### 2. BACKGROUND

The compound 1-(4-((1R,2S,3R)-1,2,3,4-tetrahydroxybutyl)-1H-imidazol-2-yl)ethanone (THI) is a minor constituent of Carmel Color III that may have therapeutic properties. See, e.g., U.S. Pat. No. 4,567,194. THI is also an intermediate in the synthesis of certain compounds that are believed to be useful in the treatment of diseases such as rheumatoid arthritis and type I diabetes. See, e.g., U.S. patent application publication nos. US-2007-0208063-A1 and US-2008-0262241-A1.

Methods of preparing THI have been reported. See, e.g., Kröplien, U. and Rosdorfer, J., J. Org. Chem. 50:1131-1133 (1985); U.S. Pat. No. 4,567,194 to Kröplien et al.; Cliff, M. D. and Pyne, S. G., Tet. Lett. 36(33):5969-5972 (1995); Cliff, M. D. and Pyne, S. G., J. Org. Chem. 62:1023-1032 (1997); Halweg, K. M. and Büchi, G., J. Org. Chem. 50:1134-1136, 1135 (1985) (“Büchi”). For example, Büchi described a method of preparing THI from glucosamine hydrochloride, although it afforded a yield of only 19%. Büchi at 1135. Consequently, better methods of preparing THI are desired.

### 3. SUMMARY OF THE INVENTION

This invention encompasses methods of preparing 1-(4-((1R,2S,3R)-1,2,3,4-tetrahydroxybutyl)-1H-imidazol-2-yl)ethanone and salts thereof. Also encompassed are methods of preparing (E)-1-(4-((1R,2S,3R)-1,2,3,4-tetrahydroxybutyl)-1H-imidazol-2-yl)-ethanone oxime and salts thereof.

### 4. DETAILED DESCRIPTION

This invention is based, in part, on the discovery of methods of preparing 1-(4-((1R,2S,3R)-1,2,3,4-tetrahydroxybutyl)-1H-imidazol-2-yl)ethanone (THI) that can afford the compound with good yields, and which are well suited for its large-scale (e.g., kilogram scale) manufacture.

### 4.1. Definitions

Unless otherwise indicated, the phrase “greater than X,” where X is a number, has the same meaning as “X or greater than X.” Similarly, the phrase “greater than about X,” where X is a number, has the same meaning as “about X or greater than about X.”

Unless otherwise indicated, the phrase “less than X,” where X is a number, has the same meaning as “X or less than X.” Similarly, the phrase “less than about X,” where X is a number, has the same meaning as “about X or less than about X.”

Unless otherwise indicated, the term “include” has the same meaning as “include” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Tautomers of compounds described herein are encompassed by the invention.

### 4.2. Methods of Synthesis

This invention encompasses methods of preparing THI:

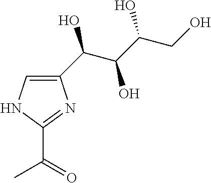

and pharmaceutically acceptable salts thereof, from 2-ethoxyacrylonitrile and D-glucosamine. Particular methods utilize a weak acid of D-glucosamine, such as D-glucosamine acetate. Applicants discovered that the use of such weak acids can afford yields of THI that exceed those reported in the literature. See, e.g., Büchi at 1135.

One embodiment encompasses a method of preparing 1-(4-((1R,2S,3R)-1,2,3,4-tetrahydroxybutyl)-1H-imidazol-2-yl)ethanone, which comprises: contacting 2-ethoxyacrylimidate with a weak acid salt of D-glucosamine to provide a first mixture; contacting the first mixture with a base to provide a second mixture; adding an aqueous acid to the second mixture to provide a third mixture; and isolating 1-(4-((1R,2S,3R)-1,2,3,4-tetrahydroxybutyl)-1H-imidazol-2-yl)ethanone from the third mixture. Examples of weak acid salts of D-glucosamine include D-glucosamine acetate. Other suitable weak acids include organic acids (e.g., formic acid, trichloroacidic acid, propionic acid, benzoic acid, citric acid, succinic acid, lactic acid) and inorganic acids (e.g., carbonic acid, phosphoric acid, and phosphonic acid).

Examples of bases include alkaline or alkaline earth metal alkoxides, hydroxides, carbonates, phosphates, trialkylamines. A particular base is methoxide.

In a particular method, the first mixture is maintained at a temperature of greater than about 10° C., 15° C., or 20° C. for at least about 0.5, 4 or 8 hours.

In a particular method, the second mixture is maintained at a temperature of greater than about 5° C., 10° C. or 20° C. for at least about 1, 2, or 3 hours.

In a particular method, the third mixture is maintained at a temperature of greater than about 20° C., 30° C. or 50° C. for at least about 0.5, 1, or 3 hours.

In a particular method, the aqueous acid has a pKₐ of from about 0 to about 10, from about 0 to about 8, or from about 0 to about 6. Examples of aqueous acids include formic, acetic, and trichloroacetic acid, hydrochloric acid, sulfuric acid, and phosphoric acid.

In a particular method, the 1-(4-((1R,2S,3R)-1,2,3,4-tetrahydroxybutyl)-1H-imidazol-2-yl)ethanone is isolated by filtering a slurry prepared by concentrating, cooling and/or diluting the third mixture with water.

Preferred methods afford 1-(4-((1R,2S,3R)-1,2,3,4-tetrahydroxybutyl)-1H-imidazol-2-yl)ethanone with a yield of greater than about 50, 55, 60, or 65 percent.

In a particular method, the 2-ethoxyacrylimidate is prepared by contacting 2-ethoxyacrylonitrile with an alcohol and an alkaline or alkaline earth metal alkoxide (e.g., sodium methoxide, sodium ethoxide) to provide a fourth mixture. In one method, the fourth mixture is maintained at a temperature of greater than about 0° C., 5° C. or 10° C. for at least about 2, 6 or 8 hours.

One embodiment of the invention encompasses a method of preparing 1-(4-((1R,2S,3R)-1,2,3,4-tetrahydroxybutyl)-1H-imidazol-2-yl)ethanone, which comprises: maintaining an initial mixture comprising D-glucosamine or a salt thereof, a weak acid or a metal salt thereof, and an alcohol solvent at a temperature of greater than about 0° C.; contacting 2-ethoxyacrylimidate with the initial mixture to provide a first mixture; contacting the first mixture with a base to provide a second mixture; contacting the second mixture with aqueous acid to provide a third mixture; and isolating 1-(4-((1R,2S,3R)-1,2,3,4-tetrahydroxybutyl)-1H-imidazol-2-yl)ethanone from the third mixture.

This invention also encompasses methods of preparing (E)-1-(4-((1R,2S,3R)-1,2,3,4-tetrahydroxybutyl)-1H-imidazol-2-yl)-ethanone oxime. In one embodiment, (E)-1-(4-((1R,2S,3R)-1,2,3,4-tetrahydroxybutyl)-1H-imidazol-2-yl)-ethanone oxime is prepared by contacting THI (prepared as described herein) with hydroxylamine or a salt thereof under conditions sufficient to form (E)-1-(4-((1R,2S,3R)-1,2,3,4-tetrahydroxybutyl)-1H-imidazol-2-yl)-ethanone oxime. Particular conditions include the use of a solvent (e.g., methanol or a mixture comprising ethanol), the presence of a base when hydroxylamine salts are used (e.g., sodium acetate, triethylamine, sodium carbonate, sodium methoxide), and optional heating (e.g., at a temperature of greater than about 40° C., 50° C. or 60° C.) for a time (e.g., greater than about 1, 2 or 4 hours) sufficient to afford (E)-1-(4-((1R,2S,3R)-1,2,3,4-tetrahydroxybutyl)-1H-imidazol-2-yl)-ethanone oxime.

### 5. EXAMPLES

Aspects of this invention can be understood from the following examples, which do not limit its scope.

### 5.1. Example 1

### Preparation of 1-(4-((1R,2S,3R)-1,2,3,4-tetrahydroxybutyl)-1H-imidazol-2-yl)ethanone

To a 1000 mL three-necked flask (R1) was charged 40.0 g (0.41 mol) of 2-ethoxyacrylonitrile and 400 mL of MeOH, and the resulting mixture was stirred at about 10-20° C. for 10 minutes. To this mixture was added slowly 26.7 g (0.12 mol) of 25.0% NaOMe/MeOH at a temperature below 20° C. This mixture was stirred for 8 hours at ambient temperature. To a 2000 mL three-necked flask (R2) was charged 88.7 g (0.41 mol) of glucosamine HCl salt, 40.6 g (0.49 mol) of NaOAc and 400 mL of MeOH, and the resulting mixture was stirred at 15-25° C. for 1 hour. The mixture in R1 was transferred slowly to R2 via a dropping funnel. This mixture was stirred at 10-25° C. for 36 hours and 53.3 g (0.36 mol) of 25.0% NaOMe/MeOH was added slowly. The resulting mixture was further stirred at 10-25° C. for 36 hours and 500 mL of water and 62.4 g (1.1 mol) of HOAc were charged. This mixture was heated to 50° C. for 3 hours and concentrated to about 400 ml, cool to 0-5° C., and stirred at this temperature for 2 hours. The product was isolated by filtration and drying under vacuum. The final product (THI) was obtained as an off-white solid (69.6 g, 74% yield, 98% purity by HPLC area). ¹H NMR (D₂O) δ 7.23 (br s, 1H), 4.90 (br s, 1H), 3.71 (br m, 2H), 3.63 (m, 1H), 2.48 (s, 3H).

### 5.2. Example 2

### Alternate Preparation of 1-(4-((1R,2S,3R)-1,2,3,4-tetrahydroxybutyl)-1H-imidazol-2-yl)ethanone

To a reactor was charged 2-ethoxyacrylonitrile (26.4 kg, 96.7% purity, 263 mol) and MeOH (188 kg) at room temperature. To the solution was added slowly NaOMe/MeOH (25 w %, 18.8 kg, 87 mol), while keeping temperature at 0-10° C. The reaction mixture was then stirred at 20-25° C. for 8 to 10 hours to generate 2-ethoxyacrylimidate.

To a separate reactor was charged D-glucosamine hydrochloride (67.5 kg, 313 mol), NaOAc (26.8 kg, 327 mol), and MeOH (188 kg). The above methanol solution of methyl 2-ethoxyacrylimidate was added slowly, while keeping temperature at 0-10° C. The reaction mixture was then stirred at 20 to 25° C. for 8 to 9 hours. The stirred mixture was cooled back to 0-10° C. and NaOMe/MeOH (25%, 35.6 kg, 165 mol) was added at this temperature. The reaction mixture was then stirred at 20 to 25° C. for 4 to 5 hours to give the enol ether precursor.

To the reaction mixture was added water (293 kg) followed by HOAc (35.0 kg, 583 mol) at 20-30° C. The resulting mixture was stirred at 20-30° C. for 30 minutes and then heated at 55-60° C. for 3 to 3.5 hours.

Seeds of 1-(4-((1R,2S,3R)-1,2,3,4-tetrahydroxybutyl)-1H-imidazol-2-yl)ethanone (0.39 kg) were added to the reaction mixture at 55-60° C., and the mixture was stirred at this temperature for 1 to 2 hours. The reaction mixture was cooled to below 40° C., concentrated under vacuum to 128-204 L, re-diluted with water (26 kg), and further concentrated under vacuum to 102-179 L. The mixture was then cooled to 0-10° C. and stirred at this temperature for 2 to 3 hours. The slurry was filtered and the wet cake was washed with cold water (26 kg). The wet cake was dried under vacuum at 45-55° C. to obtain the desired product (THI, 43.25 kg, 99.8 A % and 98.0% purities, 70.0% yield) as an off-white solid.

### 5.3. Example 3

### Preparation of (E)-1-(4-((1R,2S,3R)-1,2,3,4-Tetrahydroxybutyl)-1H-imidazol-2-yl)-ethanone Oxime Dihydrate

To a 3-neck, 3-L round bottom flask equipped with a mechanical stirrer, a temperature controller and a condenser were charged with 1-(4-((1R,2S,3R)-1,2,3,4-tetrahydroxybutyl)-1H-imidazol-2-yl)ethanone (100.0 g, 434.4 mmol), hydroxylamine hydrochloric acid salt (45.2 g, 1.5 equiv), sodium acetate (53.4 g, 1.5 equiv) and methanol (HPLC grade, 1.0 L, 10×). The above solution was heated at 65° C. with stirring for 2 hours.

To the mixture was then added a solution of HCl in isopropanol (freshly prepared by slow addition of 92.7 ml acetyl chloride to 200 ml isopropanol at 0° C., 3.0 equiv) over 15 minutes and resulting mixture stirred at 65° C. for 3 hours. The mixture was diluted with MeOH (1.0 L, 10×) and cooled to room temperature and the precipitated sodium chloride was removed by filtration. The solids were washed with MeOH (100 ml, 1×) and the solution was concentrated at 40° C. under vacuum until solids started to form (about 200 ml). Water (1.0 L, 10×) was then added and the residual organic solvents were removed at 40° C. under vacuum. A polish filtration was performed to afford a clear yellow solution. To this solution was slowly added 50% NaOH aqueous solution at room temperature so that the temperature of the mixture did not exceed 40° C., until the pH reached 7.2 (7.0-7.5). The resulting solution was then heated to 65° C. to form a homogeneous solution, and concentrated under vacuum at 65° C. (60-70° C.) until the solution reached about 500 ml (5×) overall volume. The mixture was then cooled to room temperature slowly, further cooled to 0° C., and stirred at 0° C. for 1 hour. The solids were collected by filtration and washed with water (0° C., 100 ml, 1×x2) to afford a white crystalline solid.

To the above wet solid was added water (400 ml) and the resulting mixture was heated to 70-80° C. until all dissolved. The solution was cooled to room temperature and then stirred at 0° C. for 1 hour. The solids were collected by filtration and washed with water (0° C., 100 ml, 1×x2) and then dried under vacuum at 30° C. overnight to afford 99.4 g of the title compound. NMR analysis showed that the material contained about 3% of the Z isomer.

### 5.4. Example 4

### Alternative Preparation of (E)-1-(4-((1R,2S,3R)-1,2,3,4-Tetrahydroxybutyl)-1H-imidazol-2-yl)-ethanone Oxime Dihydrate

To a reactor was charged with THI (71.65 kg, 311 mol, 1×), hydroxylamine hydrochloride (17.0 kg, 245 mol, 0.24×), sodium acetate (39.15 kg, 477 mol, 0.55×) and methanol (571 kg, 7.97×). The solution was heated at 60 to 65° C. with stirring for 3 to 4 hours and then cooled to 20 to 30° C. The mixture was charged with hydroxylamine hydrochloride (17.0 kg, 245 mol, 0.24×) and then heated to 60 to 65° C. with stirring for 9 to 10 hours, after which it was cooled to 25 to 30° C.

To the mixture was then added HCl gas (31.4 kg, 0.44×) sub-surface at a temperature below 40° C. and resulting mixture was heated at 60 to 65° C. with stirring for 3 to 4 hours. The solution was cooled to 25 to 30° C. and concentrated to about 179-215 L (2.5-3.0×) at below 50° C. under vacuum. Water (350 kg, 4.88×) was then added and the mixture was heated to 55-60° C. to dissolve all solids. The mixture was cooled to below 40° C. 10% NaOH solution (286 kg, 4.0×) was added slowly to adjust pH to 7.0-8.0. To this mixture was added charcoal (2.0 kg, 0.028×). The resulting mixture was heated at 55 to 65° C. for 50 to 60 minutes and then filtered. The wet cake was washed with hot water (46 kg, 0.6×). The filtrate was concentrated to 394-466 L (5.5-6.5×) under vacuum at a temperature below 55° C. The mixture was cooled to 30 to 40° C. over 1 to 2 hours, to 20 to 30° C. over 1 to 2 hours, to 10 to 20° C. over 1 to 2 hours, and then to 0 to 5° C. over 2 to 3 hours. The resulting suspension was stirred at 0 to 5° C. for 2 to 3 hours and filtered. The wet cake was washed with cold water (56 kg, 0.78×).

To the wet cake was added purified water (649 kg, 9.05×) and the mixture was heated to 55 to 65° C. and stirred until all solids dissolved. The hot solution was polish-filtered and rinsed with pre-warmed purified water (72 kg, 1.0×). The filtrate was concentrated to 394-466 L (5.5-6.5×) at 45 to 55° C. under vacuum. The mixture was cooled to 30 to 40° C. over 1 to 2 hours, to 20 to 30° C. over 1 to 2 hours, to 10 to 20° C. over 1 to 2 hours, and then to 0 to 5° C. over 2 to 3 hours. The resulting suspension was stirred at 0 to 5° C. for 2 to 3 hours and filtered. The wet cake was washed with cold purified water (50 kg, 0.7×). The wet cake (64.8 kg) were collected and dried under vacuum at 30 to 35° C. until KF was 12.9% to afford 62.1 kg of the title compound as a white solid (98.9 A % and 99.5% purities, 70.6% of isolated yield). ¹H NMR (D₂O w/ a drop of DCI in D₂O) 7.30 (s, 1H), 5.04 (s, 1H), 3.45-3.75 (m, 4H), 2.13 (s, 3H); ¹³C NMR (D₂O w/ a drop of DCI in D₂O) 143.8, 140.9, 135.0, 116.9, 72.5, 70.6, 64.4, 62.7, 10.5; MH⁺=246.1.

### 5.5. Example 5

### Preparation of Anhydrous (E)-1-(4-((1R,2S,3R)-1,2,3,4-Tetrahydroxybutyl)-1H-imidazol-2-yl)-ethanone Oxime

The solid from Example 3 was slurried with EtOH (800 ml, 8×) and heated at 75° C. for 1 hour. The resulting mixture was cooled to 0° C. and stirred at 0° C. for 1 hour. The white solid was collected by filtration and washed with EtOH (0° C., 100 ml, 1×, x2) and dried at 50° C. under vacuum to constant weight to give the title compound. NMR analysis showed about 2% of the Z isomer. ¹H NMR (D₂O) 7.05 (s, 1H), 4.83 (d, J=3.6 Hz, 1H), 3.60-3.80 (m, 3H), 3.50 (dd, J=11.6, 6.8 Hz, 1H), 2.11 (d, J=4.0 Hz, 3H); ¹H NMR (D₂O w/ a drop of DCI in D₂O) 7.30 (s, 1H), 5.04 (s, 1H), 3.45-3.75 (m, 4H), 2.13 (s, 3H); ¹³C NMR (D₂O w/ a drop of DCI in D₂O) 143.8, 140.9, 135.0, 116.9, 72.5, 70.6, 64.4, 62.7, 10.5; MH⁺=246.1.

All cited publications, patents, and patent applications are herein incorporated by reference in their entireties.

What is claimed is:

1. A method of preparing 1-(4-((1R,2S,3R)-1,2,3,4-tetrahydroxybutyl)-1H-imidazol-2-yl)ethanone, which comprises:
contacting 2-ethoxyacrylimidate with a weak acid salt of D-glucosamine to provide a first mixture;
contacting the first mixture with a base to provide a second mixture;
adding an aqueous acid to the second mixture to provide a third mixture; and
isolating 1-(4-((1R,2S,3R)-1,2,3,4-tetrahydroxybutyl)-1H-imidazol-2-yl)ethanone from the third mixture.

2. The method of claim 1, wherein the weak acid salt of D-glucosamine is D-glucosamine acetate.

3. The method of claim 2, wherein the base is an alkaline or alkaline earth metal alkoxide, hydroxide, carbonate, phosphate, or trialkylamine.

4. The method of claim 3, wherein the base is methoxide.

5. The method of claim 1, wherein the first mixture is maintained at a temperature of greater than 10° C. for at least 0.5 hours.

6. The method of claim 1, wherein the second mixture is maintained at a temperature of greater than 5° C. for at least 1 hour.

7. The method of claim 1, wherein the third mixture is maintained at a temperature of greater than 20° C. for at least 0.5 hours.

8. The method of claim 1, wherein the aqueous acid has a pKₐ of from 0 to 10.

9. The method of claim 8, wherein the aqueous acid is formic, acetic, or trichloroacetic acid.

10. The method of claim 9, wherein the aqueous acid is acetic acid.

11. The method of claim 1, wherein the 1-(4-((1R,2S,3R)-1,2,3,4-tetrahydroxybutyl)-1H-imidazol-2-yl)ethanone is isolated by filtering a slurry prepared by concentrating, cooling and/or diluting the third mixture with water.

12. The method of claim 1, wherein the 1-(4-((1R,2S,3R)-1,2,3,4-tetrahydroxybutyl)-1H-imidazol-2-yl)ethanone is isolated with a yield of greater than about 50 percent.

13. The method of claim 1, wherein the 2-ethoxyacrylimidate is prepared by contacting 2-ethoxyacrylonitrile with an alcohol and an alkaline or alkaline earth metal alkoxide to provide a fourth mixture.

14. The method of claim 13, wherein the fourth mixture is maintained at a temperature of greater than 0° C. for at least 2 hours.

15. A method of preparing 1-(4-((1R,2S,3R)-1,2,3,4-tetrahydroxybutyl)-1H-imidazol-2-yl)ethanone, which comprises:
contacting 2-ethoxyacrylimidate with the initial mixture at a temperature of greater than 0° C. to provide a first mixture, wherein the initial mixture comprises D-glucosamine or a salt thereof, a weak acid or a metal salt thereof, and an alcohol;
contacting the first mixture with a base to provide a second mixture;
contacting the second mixture with aqueous acid to provide a third mixture; and
isolating 1-(4-((1R,2S,3R)-1,2,3,4-tetrahydroxybutyl)-1H-imidazol-2-yl)ethanone from the third mixture.
